(19) United States
(12) Patent Application Publication
(10) Pub. No.: US 2013/0078304 A1
(43) Pub. Date: Mar. 28, 2013

(54) CONTROLLED RELEASE FORMULATION FOR TREATING SLEEP DISORDERS

(71) Applicants: HSIEN-JEN HSIEH, TAOYUAN CITY (TW); KUO-HUA YANG, TAOYUAN CITY (TW); CHIH-SHENG YANG, TAOYUAN CITY (TW); YU-CHENG WEI, TAOYUAN CITY (TW); CHUNG-HSUAN LU, TAOYUAN CITY (TW)

(72) Inventors: HSIEN-JEN HSIEH, TAOYUAN CITY (TW); KUO-HUA YANG, TAOYUAN CITY (TW); CHIH-SHENG YANG, TAOYUAN CITY (TW); YU-CHENG WEI, TAOYUAN CITY (TW); CHUNG-HSUAN LU, TAOYUAN CITY (TW)

(73) Assignee: TAIWAN BIOTECH CO., LTD., TAOYUAN CITY (TW)

(21) Appl. No.: 13/247,302

(22) Filed: Sep. 28, 2011

(51) Int. Cl. A61K 31/519; A61P 25/00; A61K 9/24; A61K 9/52; A61K 9/30; A61K 9/32

(52) U.S. Cl. 424/458; 514/259.3; 424/471; 424/472; 424/457

(57) ABSTRACT

The invention relates to a controlled-release formulation for preventing and/or treating sleep disorders comprising Zaleplon or a pharmaceutically acceptable salt thereof in immediate release form and Zolpidem or a pharmaceutically acceptable salt thereof in sustained release form, wherein Zaleplon or a pharmaceutically acceptable salt thereof and Zolpidem or a pharmaceutically acceptable salt thereof are released in two phases where the first phase is a immediate release phase of Zaleplon or a pharmaceutically acceptable salt thereof and the second phase is a sustained release phase of Zolpidem or a pharmaceutically acceptable salt thereof.

### FIELD OF THE INVENTION

The present invention relates to a controlled release formulation for treating sleep disorders, which has a modified release profile. Particularly, the invention provides a controlled release formulation comprising Zolpidem and Zaleplon.

### BACKGROUND OF THE INVENTION

Insomnia is defined as difficulty falling asleep or maintaining sleep, which interferes with a patient's daytime functioning. Insomnia is the most common sleep complaint with a prevalence of 26% to 50% in adult population.

Benzodiazepines have been the mainstay of therapy for insomnia and are available as short, intermediate or long-acting hypnotic agents. When used for a short period of time, the benzodiazepines are useful in treating insomnia. However, the benzodiazepines pose potential problems such as altering sleep architecture, rebound insomnia when discontinued, possible hangover effects and abuse, as well as development of tolerance to the drug.

The development of selective benzodiazepine₁ receptor agonists has produced two currently available compounds, Zolpidem (Ambien®, Searle and Co.) and Zaleplon (Sonata®, Wyeth-Ayerst Co.). Zolpidem and Zaleplon are non-benzodiazepine sedative agents that act selectively on benzodiazepine (BZ₁) receptors. By virtue of their short half-life, it is thought that these agents should prevent patients from experiencing benzodiazepine₂ receptor effects involving memory, cognition and psychomotor function. In the literature, neither Zolpidem nor Zaleplon is reported to affect sleep architecture as the benzodiazepines do.

Zolpidem is an imidazopyridine that binds selectively and potently to the BZ₁ receptor. It does not produce muscle-relaxant or anticonvulsant effects at doses employed for sleep. It has been demonstrated to reduce sleep latency, increase sleep duration, and reduce nighttime awakenings. Zolpidem's half-life is approximately 2.5 hours. Metabolism decreases with age, resulting in the use of doses 50% lower in the elderly. Zolpidem's advantage is that it preserves stage-IIII and stage-IV sleep and has less disruption of REM (Rapid Eye Movement) sleep. Zolpidem is poorly soluble in aqueous media.

Zaleplon is a pyrazolopyrimidine derivative that is selective for the BZ₁ receptor but is more weakly bound to the receptor than is Zolpidem. Onset of effect is reported to be slightly more rapid than that of Zolpidem. The half-life is about one hour and is not affected by aging. Zaleplon is not recommended for sleep maintenance. Zaleplon is poorly soluble in aqueous media.

It is desirable to develop a pharmaceutical formulation for oral application of rapid acting hypnotic agent that exhibits a fast but also prolonged action. Delayed/sustained release compositions and dosage forms, namely those in which a specific agent or device is present to act as a means for controlling the release rate of an active substance, are well known in the prior art. However, such conventional delayed/sustained release formulations are generally contrary to the purpose of a rapid acting hypnotic. The patient taking Zolpidem desires the onset of the sleeping effect to be rapid. But a conventional sustained release formulation would delay the onset of sleep. Accordingly, the use of traditional release modifying agents such as acrylate polymers would be expected to be inconsistent with the administration of a simple, rapid acting hypnotic dosage form. Attempts have also been made to provide controlled-release dosage forms, particularly in the context of Zolpidem and salts thereof. US 20060159744 relates to timed dual release dosage forms of short acting hypnotics (such as zolpidem) or salts thereof adapted to release the short acting hypnotic over a predetermined time period, according to a profile of dissolution characterized in that it comprises two release pulses, the first being immediate (lasting up to 30 minutes) and the second being delayed by a fixed time (this fixed time being between 50 and 200 minutes. US 2009/0156631 discloses a controlled release composition of Zolpidem or pharmaceutically acceptable salts thereof adapted to release Zolpidem over a predetermined time period, according to a monophasic and/or a biphasic profile of dissolution. US 2007/0231381 discloses a controlled-release zolpidem composition comprising granules that comprise Zolpidem or a salt thereof, a water-insoluble polymer, and an enteric polymer. The above prior art references illustrate controlled-release profile of single short acting hypnotic and attempt use the controlled release means to improve the defects of short acting hypnotic. However, a satisfying effect has not been achieved yet.

Therefore, there is a need in the art for sedative-hypnotic compositions that induce and maintain sleep but without the side effects associated with the longer acting hypnotics.

### SUMMARY OF THE INVENTION

The invention provides a controlled-release formulation comprising Zaleplon or a pharmaceutically acceptable salt thereof in immediate release form and Zolpidem or a pharmaceutically acceptable salt thereof in sustained release form.

### BRIEF DESCRIPTION OF THE DRAWING

FIG. 1 shows the in vitro dissolution profile of the sustained release phase of Zolpidem of the controlled-release tablet of example 1, where the total amount of Zolpidem dissolved in 5 hours.

FIG. 2 shows the in vitro dissolution profile of the immediate release phase of Zaleplon of the controlled-release tablet of example 1, where over 80% Zaleplon dissolved in 15 minutes and the total amount of Zaleplon dissolved in 1 hour.

FIG. 3 shows the in vitro dissolution profile of the immediate release phase of Zaleplon, where over 80% Zaleplon dissolved in 15 minutes and the total amount of Zaleplon dissolved in 1 hour of the controlled-release double layer table.

FIG. 4 shows the in vitro dissolution profile of the sustained release phase of Zolpidem of the controlled-release double layer table, where the total amount of Zolpidem dissolved in 5 hours.

FIG. 5 shows the in vitro dissolution profile of the immediate release phase of Zaleplon, where over 80% Zaleplon dissolved in 15 minutes and the total amount of Zaleplon dissolved in 1 hour of the controlled-release capsule of Example 3.

FIG. 6 shows the in vitro dissolution profile of the sustained release phase of Zolpidem of the controlled-release capsule of Example 3, where the total amount of Zolpidem dissolved in 5 hours.

FIG. 7 shows the in vitro dissolution profile of the immediate release phase of Zaleplon, where over 80% Zaleplon dissolved in 15 minutes and the total amount of Zaleplon dissolved in 1 hour of the controlled-release capsule of Example 4.

FIG. 8 shows the in vitro dissolution profile of the sustained release phase of Zolpidem of the controlled-release capsule of Example 4, where the total amount of Zolpidem dissolved in 5 hours.

### DETAILED DESCRIPTION OF THE INVENTION

The invention surprisingly found that a specific combination of Zaleplon in immediate release form and Zolpidem in sustained release form can produce a controlled-release hypnotic formulation having an unexpectable efficacy in inducing and maintaining sleep in a sufficient period without the side effects such as discontinuous sleep and headache. The immediate release Zaleplon brings on sleep more rapidly and acts for a shorter period of time, whereas the sustained release Zolpidem prolongs the sleep and is effective in the deep sleep period, so the invention can solve problems associated with sleep disorders, such as hard to fall asleep or bad sleeping but without the side effect of headache caused by persistent type hypnotics and short period of sleep caused by fugitive type hypnotics.

The term “controlled release” refers to a drug-containing formulation or fraction thereof in which release of the drug is not immediate, i.e., with a “controlled release” formulation, administration does not result in fast or immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release formulations.

The term “sustained release” is used in its conventional sense to refer to a drug formulation in which there is a continual release of the drug over a period of time following administration.

As used in this specification and the intended claims, the singular forms “a,” “an,” and “the” include plural referents unless the context clearly dictates otherwise.

The term “dissolution” is used herein to refer to the reduction of a solid dosage form of the present invention to a liquid form. More particularly, a complete dissolution of a solid dosage form refers to less than about 25% by weight of the solid dosage form remaining in the mouth following an appropriate time period, e.g., 5 minutes or less, after administration. Suitable methods known in the art for determining the dissolution profile of a solid dosage form include, e.g., USP dissolution tests such as USP <711> Apparatus 1 or USP <711> Apparatus 2.

The term “insomnia” refers to a sleep disorder characterized by symptoms including, without limitation, difficulty in falling asleep, difficulty in staying asleep, intermittent wakefulness, and/or waking up too early. The term also encompasses daytime symptoms such as sleepiness, anxiety, impaired concentration, impaired memory, and irritability. Types of insomnia suitable for treatment with the compositions of the present invention include, without limitation, transient, short-term, and chronic insomnia.

The term “sleep disorder” refers to a disruptive pattern of sleep arising from many causes including, without limitation, dysfunctional sleep mechanisms, abnormalities in physiological functions during sleep, abnormalities of the biological clock, and sleep disturbances that are induced by factors extrinsic to the sleep process. In particular, the term encompasses disorders associated with difficulties in staying asleep and/or falling asleep such as insomnia (e.g., transient, short-term, and chronic), delayed sleep phase syndrome, hypnotic-dependent sleep disorder, and stimulant-dependent sleep disorder; disorders associated with difficulties in staying awake such as sleep apnea, narcolepsy, restless leg syndrome, obstructive sleep apnea, central sleep apnea, idiopathic hypersomnia, respiratory muscle weakness-associated sleep disorder; disorders associated with difficulties in adhering to a regular sleep schedule such as sleep state misperception, shift work sleep disorder, chronic time zone change syndrome, and irregular sleep-wake syndrome; disorders associated with abnormal behaviors such as sleep tenor disorder (i.e., parasomnia) and sleepwalking (i.e., somnambulism); and other disorders such as sleep bruxism, fibromyalgia, and nightmares.

The term “administering” refers to administration of the compositions of the present invention to the body.

“Pharmaceutically acceptable salt” includes, but is not limited to, amino acid salts, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Pharmaceutically acceptable excipient or carrier” refers to an excipient that may optionally be included in the compositions of the invention and that causes no significant adverse toxicological effects to the patient.

The invention provides a controlled-release formulation comprising Zaleplon or a pharmaceutically acceptable salt thereof in immediate release form and Zolpidem or a pharmaceutically acceptable salt thereof in sustained release form. According to one embodiment of the invention, Zaleplon or a pharmaceutically acceptable salt thereof and Zolpidem or a pharmaceutically acceptable salt thereof are released in two phases, wherein according to a biphasic in vitro profile of dissolution when measured in a dissolution apparatus in about 0.1 N hydrochloric acid buffer at about 37° C.), the first phase is a immediate release phase of Zaleplon or a pharmaceutically acceptable salt thereof that is released more than about 70% within about 60 minutes, and the second phase is a sustained release phase of Zolpidem or a pharmaceutically acceptable salt thereof that is completely released between about 2 and about 6 hours.

According to the invention, Zaleplon or a pharmaceutically acceptable salt thereof used in the invention is in an immediate release form that is released in the first phase of the release profile of the formulation of the invention. Zaleplon is known as N-[3-(3-cyanopyrazolo-[1,5-a]-pyrimidin-7-yl)-phenyl]-N-ethyl acetamide. Any form of Zaleplon is suitable for use in the compositions described herein, e.g., a salt form of Zaleplon, a free base form of Zaleplon, or a mixture thereof. Preferably, Zaleplon is in free base form. The first release phase (immediate release phase of Zaleplon is the part of the dissolution profile mainly from about 0 to about 60 minutes in a suitable in vitro dissolution test; preferably, about 0 to about 40 minutes, about 0 to about 30 minutes, about 10 to about 60 minutes, about 10 to about 40 minutes or about 10 to about 30 minutes. A suitable dissolution test is for example one of the method described as follows: method where measurement is carried out in a dissolution apparatus in aqueous buffer at about 37° C., or variations on this as well known to one who is skilled in the art. In an advantageous embodiment of the dosage forms according to the present invention about 70% or more (preferably about 80% or more) of that part of the Zaleplon allotted for the first phase is dissolved in about 30 minutes (preferably about 20 or about 15 minutes) and about 100% of Zaleplon is dissolved within 45 to 120 minutes.

The immediate release Zaleplon shall be understood in the present invention as a single pharmaceutical immediate release unit like for example an immediate release layer, tablet, pellet, coating or several such units formulated into capsules, tablets or beads; as an immediate release matrix in tablet; as an immediate release layer, that can be incorporated in multilayer tablet; as an immediate release coating layer in (multi) coated tablet or pellet or beads encapsulated in a capsule.

According to the invention, Zolpidem or a pharmaceutically acceptable salt thereof used in the invention is in a sustained release form that is released in the second phase of the release profile of the formulation of the invention. Zolpidem is known as 2-(6-methyl-2-p-tolylimidazo[1,2-a]pyridin-3-yl)acetamide. Any form of Zolpidem is suitable for use in the compositions described herein, e.g., a salt form of zolpidem (e.g., zolpidem tartrate), a free base form of zolpidem, or a mixture thereof. Zolpidem tartrate is the preferred species. The second release phase (sustained release phase) is the part of the dissolution profile which is mainly after about 30 minutes, measured in a suitable in vitro dissolution test, such as described above. Zolpidem in the formulation of the invention can be completely released in the dissolution time between about 2 and about 6 hours, and preferably between about 4 and about 5 hours. The sustained release Zolpidem shall be understood in the present invention as a pharmaceutical sustained release unit such as, for example, a sustained release layer, core, tablet or pellet, or several such units formulated into a capsule or a tablet; as a sustained release layer, that can be incorporated in a multilayer tablet; as a sustained release core or a sustained release coating layer in a multicoated tablet; as sustained release pellets within a disintegrating tablet.

According to the invention, the sustained release form of Zolpidem comprises a copolymer to achieve the sustained release effect. In an embodiment of the invention, the copolymer is in an amount higher than 5% by weight and it is a member selected from hydrogels, gelatin, low molecular weight polyethylene oxides, e.g., less than 100,000 MW; hydroxyalkylcelluloses, e.g., hydroxyethylcellulose, hydroxypropylcellulose, hydroxyisopropylcelluose, hydroxybutylcellulose and hydroxyphenylcellulose; hydroxyalkyl alkylcelluloses, e.g., hydroxypropyl methylcellulose; hydroxypropyl cellulose; hydroxypropylmethyl cellulose; polyethylene oxide (such as that having a weight average molecular weight of 100,000 to 7,000,000); poly(hydroxy alkyl methacrylate) (such as that having a molecular weight of from 30,000 to 5,000,000); poly(vinyl)alcohol (such as that having a low acetal residue, which is cross-linked with glyoxal, formaldehyde or glutaraldehyde and having a degree of polymerization of from 200 to 30,000); a mixture of methyl cellulose, cross-linked agar and carboxymethyl cellulose; a hydrogel forming copolymer produced by forming a dispersion of a finely divided copolymer of maleic anhydride with styrene, ethylene, propylene, butylene or isobutylene cross-linked with saturated cross-linking agent per mole of maleic anyhydride in the copolymer; Carbopol™ acidic carboxy polymers (such as that having a molecular weight of 450,000 to 4,000,000); Cyanamer™ polyacrylamides; cross-linked water swellable indenemaleic anhydride polymers; Goodrite™ polyacrylic acid (such as that having a molecular weight of 80,000 to 200,000); starch graft copolymers; Aqua-Keeps™ acrylate polymer polysaccharides composed of condensed glucose units such as diester cross-linked polyglucan and the like and mixtures thereof. Preferably, the copolymer is in an amount from about 5% (w/w) to about 30% (w/w), about 5% (w/w) to about 25% (w/w), about 5% (w/w) to about 20% (w/w), about 5% (w/w) to about 15% (w/w), about 10% (w/w) to about 30% (w/w), about 10% (w/w) to about 25% (w/w), about 10% (w/w) to about 20% (w/w), about 10% (w/w) to about 15% (w/w), about 12% (w/w) to about 20% (w/w), about 12% (w/w) to about 17% (w/w) or about 12% (w/w) to about 15% (w/w).

According to the invention, the immediate release Zaleplon in the first phase induces the immediate sleep of the patient and the sustained release Zolpidem in the second phase allows the drug blood level to be maintained with the objective of maintaining sleep.

The immediate release Zaleplon according to the invention typically contain from about about 2 to about 10 mg of Zaleplon, and preferably about 2 to about 8 mg, about 2 to about 6 mg, about 3 to about 8 mg, and more preferably about 5 mg of Zaleplon. The sustained release Zolpidem according to the invention typically contain from about 3 to about 15 mg of Zolpidem, and preferably about 3 to about 12 mg, about 3 to about 10 mg, about 3 to about 8 mg, about 4 to about 12 mg, about 4 to about 10 mg, about 5 to about 15 mg, about 5 to about 10 mg and more preferably about 6.25 mg of Zolpidem. The Zolpidem or Zaleplon may be incorporated as the base, or as a pharmaceutically acceptable salt thereof.

Various formulations, not limiting the scope of the present invention, illustrating the invention are described hereafter.
(1.) A controlled-release tablet comprises a sustained release Zolpidem as a core coated with an immediate release layer of Zaleplon.
(2.) A controlled-release double layer tablet comprises a layer of sustained release Zolpidem and a layer of immediate release Zaleplon.
(3.) A controlled-release tablet with more than two layers comprises (i) one or two more layers of sustained release Zolpidem and (ii) one or two more layers of immediate release Zaleplon.
(4.) A controlled-release capsule comprises a core pellet of sustained release Zolpidem coated immediate release Zaleplon.
(5.) A controlled-release capsule comprises a pellet of sustained release entity of Zolpidem and a pellet of immediate release entity of Zaleplon.
(6.) A controlled-release capsule comprises a number of beads; each bead comprises a sustained release Zolpidem as a core coated with an immediate release layer of Zaleplon.

According to one embodiment of the invention, the sustained release Zolpidem in any of the above formulation is further coated with at least one release-slowing intermediate layer of slightly soluble intermediate layer to further controlled release of Zolpidem.

In an embodiment, the sustained release Zolpidem of the controlled-release formulation can further mix with a binder. The binder is added to increase the mechanical strength of the granules and tablets during formation. Binders can be added to the formulation in different ways: (1) as a dry powder, which is mixed with other ingredients before wet agglomeration, (2) as a solution, which is used as agglomeration liquid during wet agglomeration, and is referred to as a solution binder, and (3) as a dry powder, which is mixed with the other ingredients before compaction. In this form the binder is referred to as a dry binder. Solution binders are a common way of incorporating a binder into granules. In certain embodiments, the binder used in the formulation is in the form of a dry powder binder. Non-limiting examples of binders useful for the core include hydrogenated vegetable oil, castor oil, paraffin, higher aliphatic alcohols, higher aliphatic acids, long chain fatty acids, fatty acid esters, wax-like materials such as fatty alcohols, fatty acid esters, fatty acid glycerides, hydrogenated fats, hydrocarbons, normal waxes, stearic acid, stearyl alcohol, hydrophobic and hydrophilic polymers having hydrocarbon backbones, and mixtures thereof. Specific examples of water-soluble polymer binders include modified starch, gelatin, polyvinylpyrrolidone, cellulose derivatives (such as for example hydroxypropyl methylcellulose (HPMC) and hydroxypropyl cellulose (HPC)), polyvinyl alcohol and mixtures thereof. In an embodiment, the binder is HPMC. In a preferable embodiment, the binder is HPMC K100LV. In a preferable embodiment, the binder can be present in an amount of from about 1% to about 25% by weight of the formulation.

In an embodiment, the immediate release Zaleplon can be further coated with a coating. In one embodiment the coating formulations contain polymeric ingredients. It is contemplated that other excipients consistent with the objects of the present invention can also be used in the coating. In other embodiments of the formulation, polymethacrylate acrylic polymers can be employed as coating polymers. In at least one embodiment, the coating is an acrylic resin lacquer used in the form of an aqueous dispersion, such as that which is commercially available from Rohm Pharma under the trade name EUDRAGIT® or from BASF under the trade name KOLLICOAT®. In a more preferable embodiments, EUDRAGIT® E100 is used as the coating polymer, which is a cationic copolymer based on dimethylaminoethyl methacrylate and neutral methacrylic esters having a average molecular weight is approximately 150,000. Different coating polymers of the certain embodiments can be mixed together in any desired ratio in order to ultimately obtain a coating having a desirable drug dissolution profile. Coating methods can consist in spraying a solution of the polymer on the tablets, either in a pan coater or a fluid bed coating apparatus. The solvent may be organic or aqueous, depending on the nature of the polymer used. In a preferable embodiment, the solvent is alcohol. Coating methods are well known in the art.

In another embodiment of the invention the sustained release formulation comprises at least one disintegrant. Non-limiting examples of disintegrants for use in the formulation include croscarmellose sodium, crospovidone, alginic acid, sodium alginate, methacrylic acid DVB, cross-linked PVP, microcrystalline cellulose, polacrilin potassium, sodium starch glycolate, starch, pregelatinized starch and mixtures thereof. In at least one embodiment the disintegrant is selected from microcrystalline cellulose (e.g. Avicel PH101), cross-linked polyvinylpyrrolidone (e.g. KOLLIDON® CL), cross-linked sodium carboxymethylcellulose (e.g. AC-DI-SOL™), starch or starch derivatives such as sodium starch glycolate (e.g. EXPLOTAB®), or combinations with starch (e.g. PRIMOJEL™), swellable ion-exchange resins, such as AMBERLITE™ IRP 88, formaldehyde-casein (e.g. ESMA SPRENG™), and mixtures thereof. In at preferable embodiment the disintegrant is microcrystalline cellulose.

Lubricants can be added to pharmaceutical formulations to decrease any friction that occurs between the solid and the die wall during tablet manufacturing. High friction during tabletting can cause a series of problems, including inadequate tablet quality (capping or even fragmentation of tablets during ejection, and vertical scratches on tablet edges) and may even stop production. Accordingly, lubricants are added to certain tablet formulations of the present invention including certain embodiments of the formulation described herein. Non-limiting examples of lubricants useful for the core include glyceryl behenate, stearic acid, hydrogenated vegetable oils (such as hydrogenated cottonseed oil (STEROTEX®), hydrogenated soybean oil (STEROTEX® HM) and hydrogenated soybean oil & castor wax (STEROTEX® K), stearyl alcohol, leucine, polyethylene glycol (MW 1450, suitably 4000, and higher), magnesium stearate, glyceryl monostearate, stearic acid, polyethylene glycol, ethylene oxide polymers (for example, available under the registered trademark CARBOWAX® from Union Carbide, Inc., Danbury, Conn.), sodium lauryl sulfate, magnesium lauryl sulfate, sodium oleate, sodium stearyl fumarate, DL-leucine, colloidal silica, mixtures thereof and others as known in the art.

Sweeteners that can also be used in the taste-masking coating of certain embodiments of the matrix dosage forms include glucose (corn syrup), dextrose, invert sugar, fructose, and mixtures thereof (when not used as a carrier); saccharin and its various salts, such as sodium salt; dipeptide sweeteners such as aspartame; dihydrochalcone compounds, glycyrrhizin; Steva Rebaudiana (Stevioside); chloro derivatives or sucrose such as sucralose; and sugar alcohols such as sorbitol, mannitol, xylitol, and the like. Also contemplated are hydrogenated starch hydrolysates and the synthetic sweeteners such as 3,6-dihydro-6-methyl-1-1-1,2,3-oxathiazin-4-1-2,2-dioxide, particularly the potassium salt (acesulfame-K), and sodium and calcium salts thereof. The sweeteners can be used alone or in any combination thereof.

The controlled-release formulation of the present invention can further contain one or more pharmaceutically acceptable excipients such as granulating aids or agents, colorants, flavorants, pH adjusters, anti-adherents, glidants and like excipients conventionally used in pharmaceutical compositions. In an embodiment, a coloring excipient can be advantageously added as giving rise to visual change preventing abuse. It can color simultaneously the liquid or the particles or one independently of the other. Among suitable coloring excipients the following may be cited: indigotine, cochineal carminic acid, yellow orange S, allura red AC, iron oxides, cucurmin, riboflavin, tartrazine, quinoline yellow, azorubine, amaranth, carmines, erythosine, red 2G, patented blue V, glittering blue FCF, chlorophylls, copper complexes of chlorophylls, green S, caramel, glittering black BN, carbo medicinalis vegetabilis, brown FK and HT, carotenoids, Annatto extracts, paprika extracts, lycopene, lutein, canthaxanthin, beetroot red, anthocyanes, calcium carbonate, titanium dioxide, aluminium, silver, gold or litholrubin BK or any other coloring excipient suitable for an oral administration.

The examples which follow illustrate the invention without limiting it.

### EXAMPLE

### Example 1

### Controlled-Release Tablet Comprising Sustained Release Zolpidem as a Core Coating with Immediate Release Zaleplon

The materials PVP-K30, Zolpidem tartrate, lactose monohydrate, HPMC K100 LV, A vicel PH101 and tartaric acid in the below table were mixed together, granulated with water, dried and calibrated. The granules were then mixed with magnesium stearate and compressed to a tablet to obtain sustained release Zolpidem.

| | | |
|---|---|---|
| PVP-K 30 | 1.47% | (w/w) |
| Zolpidem tartrate | 2.88% | (w/w) |
| Zaleplon | 2.30% | (w/w) |
| Lactose monohydrate | 49.29% | (w/w) |
| HPMC K100 LV | 15.67% | (w/w) |
| Avicel PH101 | 14.75% | (w/w) |
| Tartaric acid | 7.37% | (w/w) |
| Mg Stearate | 0.74% | (w/w) |
| Eudragit E100 | 5.12% | (w/w) |
| Titanium Dioxide | 0.39% | (w/w) |
| Iron oxide Red | 0.02% | (w/w) |
| Total | 100 | (w/w) |

The sustained release Zolpidem is coated with the immediate release Zaleplon (containing Zaleplon, Eudragit E100, titanium dioxide and iron oxide Red) to obtain the title controlled-release tablet.

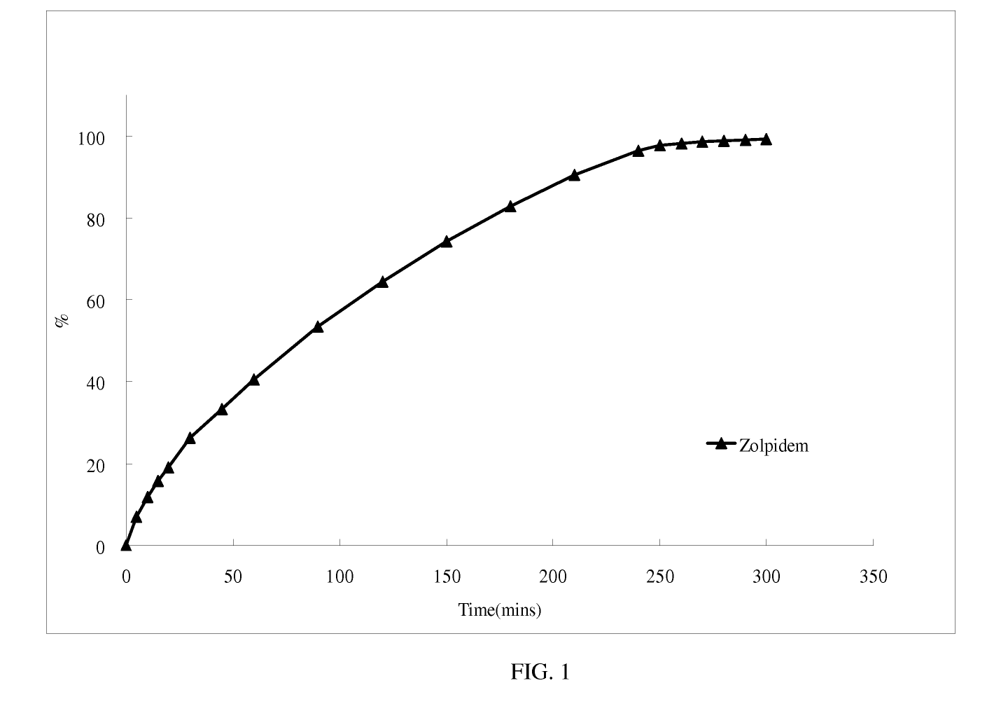

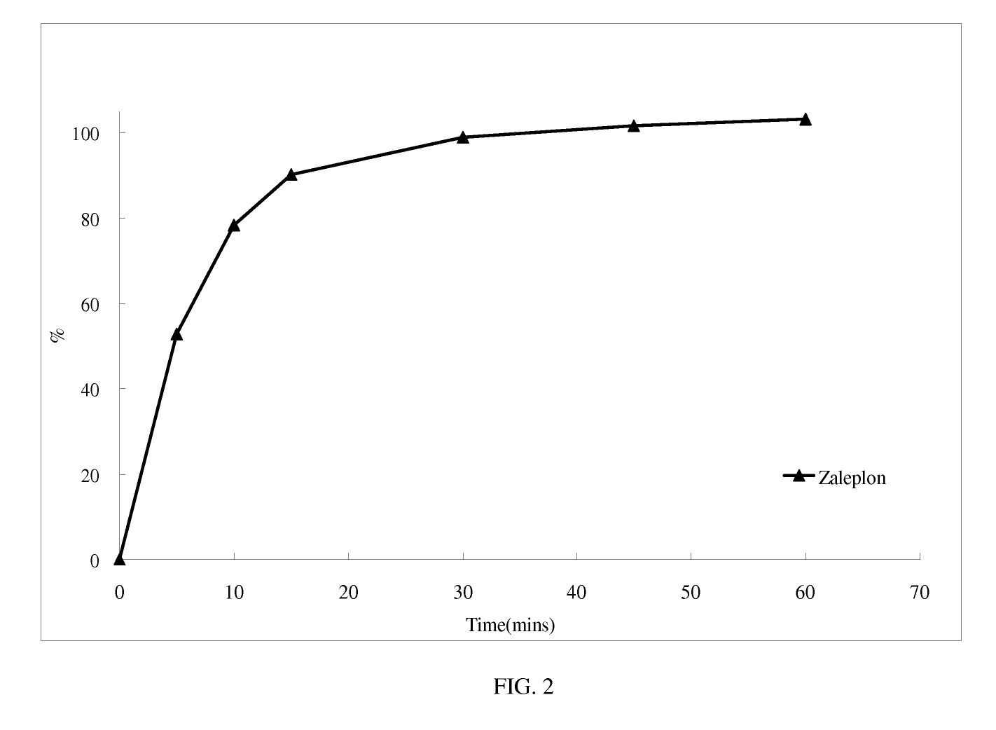

The in vitro dissolution profiles of the tablets were established using the Apparatus II of the United States Pharmacopoeia. The following dissolution medium was employed: 1000 ml 0.1N hydrochloric acid maintained at 37±0.5° C. Stirring was performed by paddle method (50 rpm). The percentage dissolved was determined by measurement of High Performance Liquid Chromatography (HPLC) at 238 nm. The results were shown in FIG. 1. and FIG. 2. FIG. 1 shows the in vitro dissolution profile of the sustained release phase of Zolpidem, where the total amount of Zolpidem dissolved in 5 hours. FIG. 2 shows the in vitro dissolution profile of the immediate release phase of Zaleplon, where over 80% of Zaleplon dissolved in 15 minutes and the total amount of Zaleplon dissolved in 1 hour.

### Example 2

### Controlled-Release Double Layer Tablet Comprising Sustained Release Zolpidem and Immediate Release Zaleplon

The formulation of the controlled-release tablet in a double-layer form is listed in the below table. Sustained release entity of the tablet comprising 6.25 mg of Zolpidem was produced as follows. The materials PVP-K 30, Zolpidem tartrate, lactose monohydrate, HPMC K100 LV, Avicel PH101 and tartaric acid were mixed together, granulated with water, dried and calibrated. The resulting granules were mixed with magnesium stearate and then pre-compressed to a sustained release Zolpidem.

| | | |
|---|---|---|
| PVP-K 30 | 1.61% | (w/w) |
| Zolpidem tartrate | 2.88% | (w/w) |
| Zaleplon | 2.30% | (w/w) |
| Lactose monohydrate | 68.07% | (w/w) |
| HPMC K100 LV | 8.30% | (w/w) |
| Avicel PH101 | 6.45% | (w/w) |
| Tartaric acid | 4.61% | (w/w) |
| Croscarmellose sodium | 1.38% | (w/w) |
| Sodium lauryl sulfate | 0.21% | (w/w) |
| Mg Stearate | 0.97% | (w/w) |
| Eudragit E100 | 2.81% | (w/w) |
| Titanium Dioxide | 0.39% | (w/w) |
| Iron oxide Red | 0.02% | (w/w) |
| Total | 100 | (w/w) |

Immediate release entity of the tablet was produced by mixing Zaleplon, lactose and PVP K30. The resulting mixture was granulated with water, dried and calibrated. The granules were then mixed with croscarmellose sodium, sodium lauryl sulfate and magnesium stearate and pre-compressed to an immediate release Zaleplon. The resulting sustained release Zolpidem and immediate release Zaleplon were compressed to obtain controlled-release double layer tablet. Eudragit E100, titanium dioxide and iron oxide Red were mixed in water and then coated on the surface of the double layer tablet.

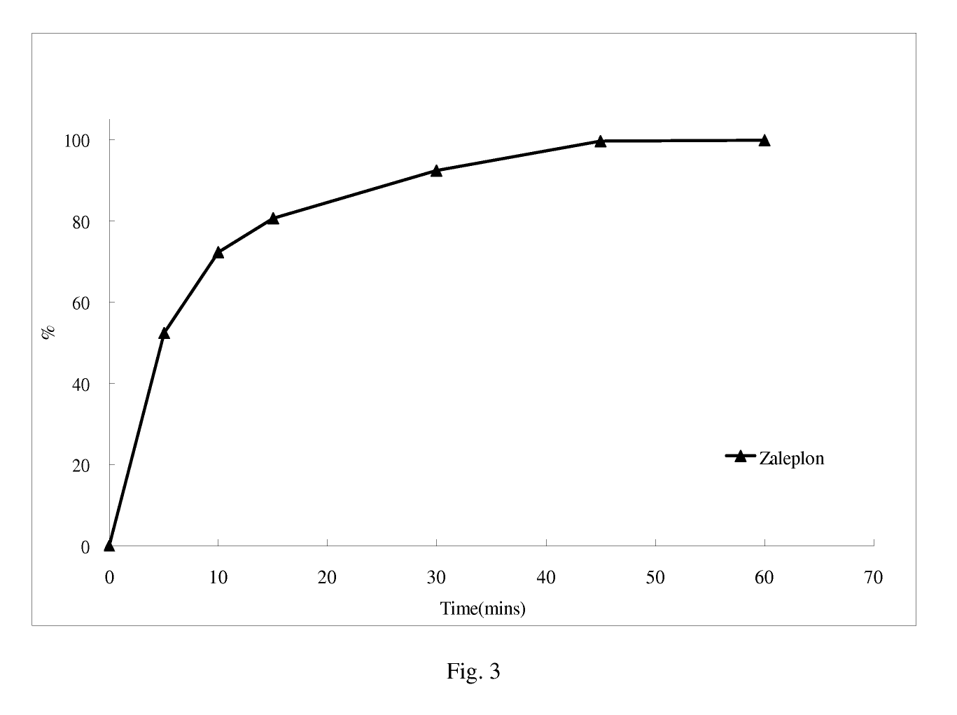

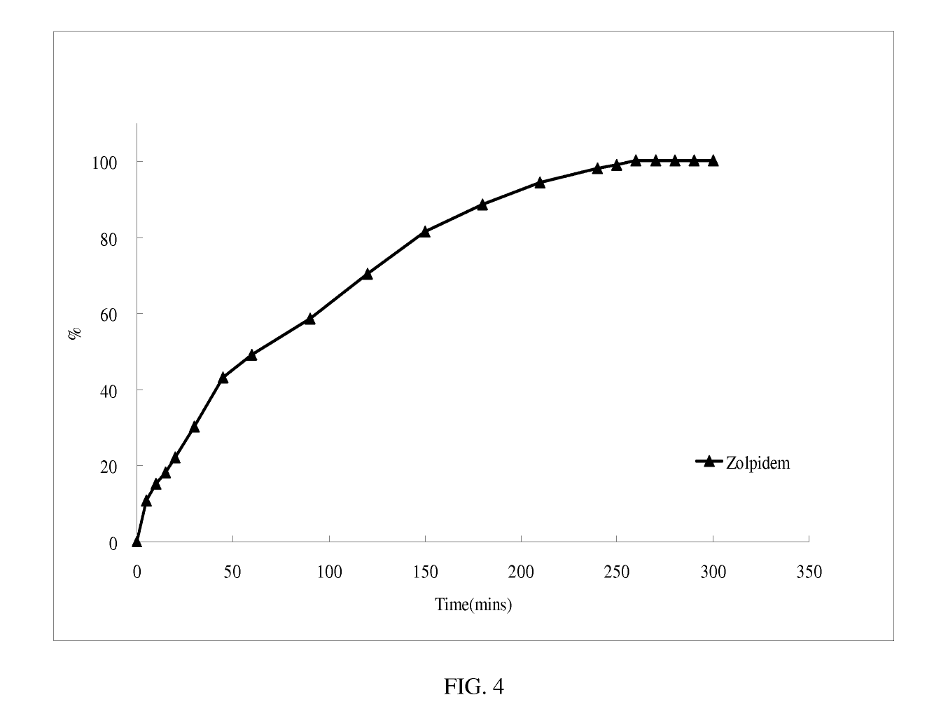

The dissolution results are also shown in FIG. 3. and FIG. 4. FIG. 3 shows the in vitro dissolution profile of the immediate release phase of Zaleplon, where over 80% of Zaleplon dissolved in 15 minutes and the total amount of Zaleplon dissolved in 1 hour. FIG. 4 shows the in vitro dissolution profile of the sustained release phase of Zolpidem, where the total amount of Zolpidem dissolved in 5 hours.

### Example 3

### Controlled-Release Capsule Comprising Pellets Comprising Sustained Release Zolpidem as a Core Coating with Immediate Release Zaleplon

Zolpidem, tartaric acid and PVP K30 were dissolved in 95% alcohol and then meshed with #100 mesh. CF granulator was used to coat the above mentioned mixture to sugar sphere #25-30 to get pellet 1. Pellet 1 was cured at 40° C. for 8 hours to reduce water content to less than 1%.

Ethycellulose N10F, PVP K30 and Triethylcitrate were dissolved in 95% alcohol and then meshed with #100 mesh. CF granulator was used to coat the mixture onto the above-mentioned pellet 1 to get pellet 2. Pellet 2 was cured at 40° C. for 8 hours to reduce water content to less than 1%.

Zaleplon and Eudragit E100 were dissolved in 95% alcohol and dichromethane and then meshed with #100 mesh. CF granulator was used to coat the above mentioned mixture onto pellet 2 to get pellet 3. Pellet 3 was cured at 40° C. for 8 hours to reduce water content to less than 1% and then put into a capsule.

The formulation of the controlled-release capsule is listed in the below table.

| | | |
|---|---|---|
| Zolpidem tartrate | 3.32% | (w/w) |
| Zaleplon | 2.66% | (w/w) |
| PVP K30 | 3.86% | (w/w) |
| Sugar Sphere #25-30 | 79.79% | (w/w) |
| Tartaric acid | 0.27% | (w/w) |
| Eudragit E100 | 2.66% | (w/w) |
| Ethylcellulose N10F | 6.38% | (w/w) |
| Triethylcitrate | 1.06% | (w/w) |
| Total | 100 | (w/w) |

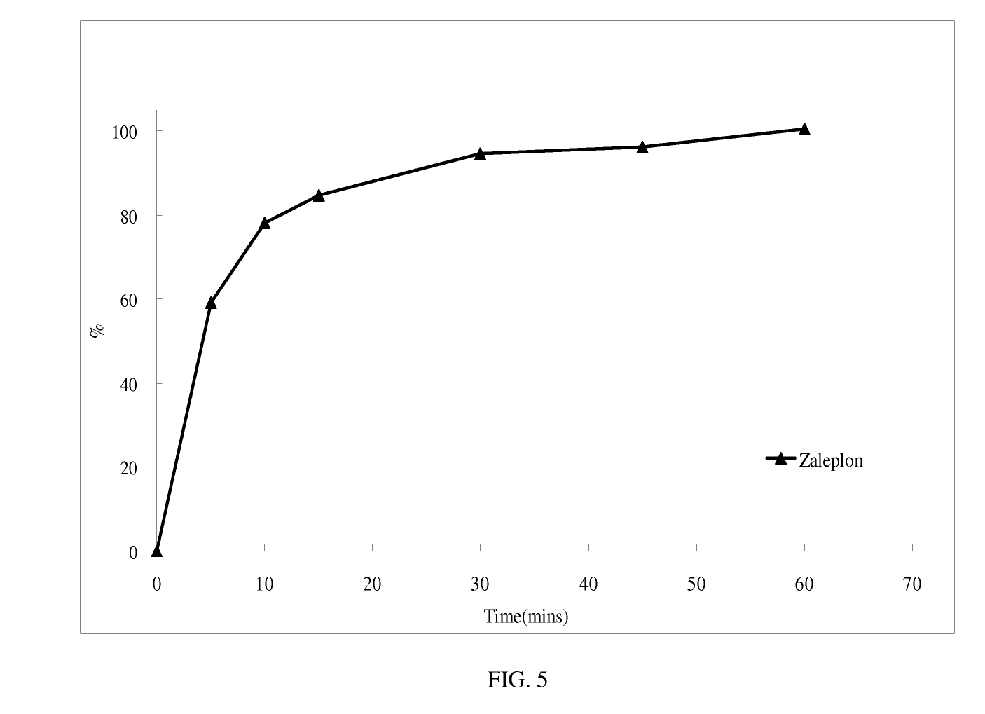

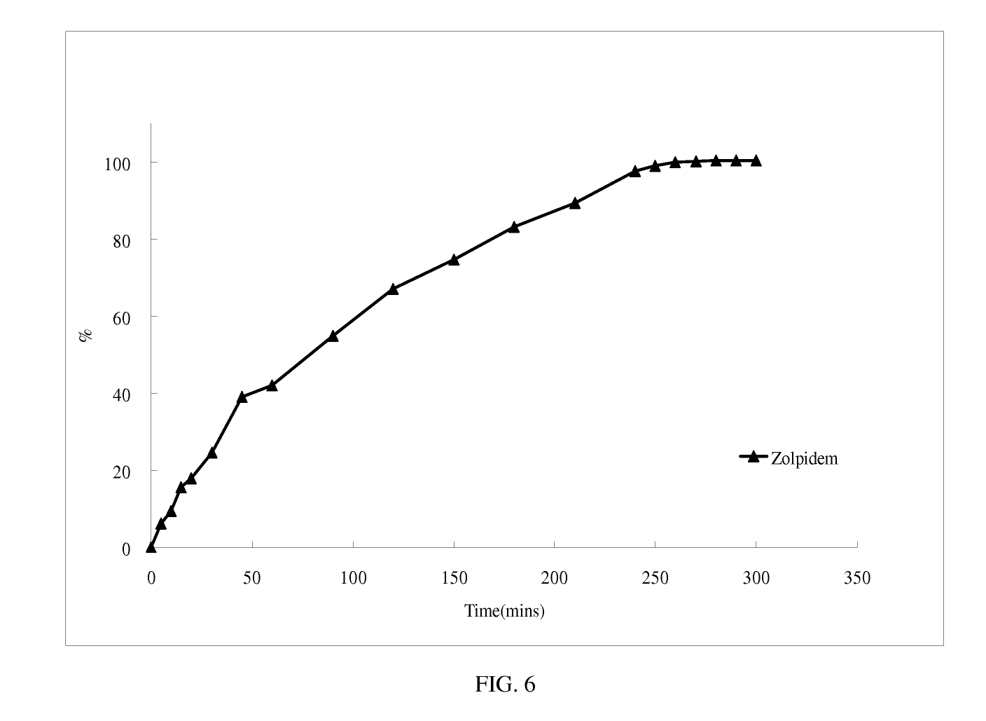

The dissolution results are also shown in FIG. 5. and FIG. 6. FIG. 5 shows the in vitro dissolution profile of the immediate release phase of Zaleplon, where over 80% Zaleplon dissolved in 15 minutes and the total amount of Zaleplon dissolved in 1 hour. FIG. 6 shows the in vitro dissolution profile of the sustained release phase of Zolpidem, where the total amount of Zolpidem dissolved in 5 hours.

### Example 4

### Controlled-Release Capsule Comprising Sustained Release Pellets of Zolpidem and Immediate Release Pellets of Zaleplon Respectively

Zolpidem, Tartaric acid and PVP K30 were dissolved in 95% alcohol and then meshed with #100 mesh. CF granulator was used to coat the above mentioned mixture onto sugar sphere #25-30 to get pellet 1. Pellet 1 was cured at 40° C. for 8 hours to reduce water content to less than 1%.

Ethycellulose N10F, PVP K30 and Triethylcitrate were dissolved in 95% alcohol and then meshed with #100 mesh. CF granulator was used to coat the mixture onto the above mentioned pellet 1 to get pellet 2. Pellet 2 was cured at 40° C. for 8 hours to reduce water content to less than 1%.

Zaleplon and Eudragit E100 were dissolved in 95% alcohol and dichloromethane and then meshed with #100 mesh. CF granulator was used to coat the above mentioned mixture onto cellet 500 to get pellet 3. Pellet 3 was cured at 40° C. for 8 hours to reduce water content to less than 1%. Capsules were then filled with Pellet 2 and Pellet 3.

The formulation of the controlled-release capsule is listed in the below table.

| | | |
|---|---|---|
| Zolpidem tartrate | 2.50% | (w/w) |
| Zaleplon | 2.00% | (w/w) |
| Eudragit E100 | 2.00% | (w/w) |
| PVP K30 | 2.90% | (w/w) |
| Sugar Sphere #25-30 | 52.80% | (w/w) |
| Tartaric acid | 0.20% | (w/w) |
| Ethylcellulose N10F | 4.80% | (w/w) |
| Triethylcitrate | 0.80% | (w/w) |
| Cellets 500 | 32% | (w/w) |
| Total | 100 | (w/w) |

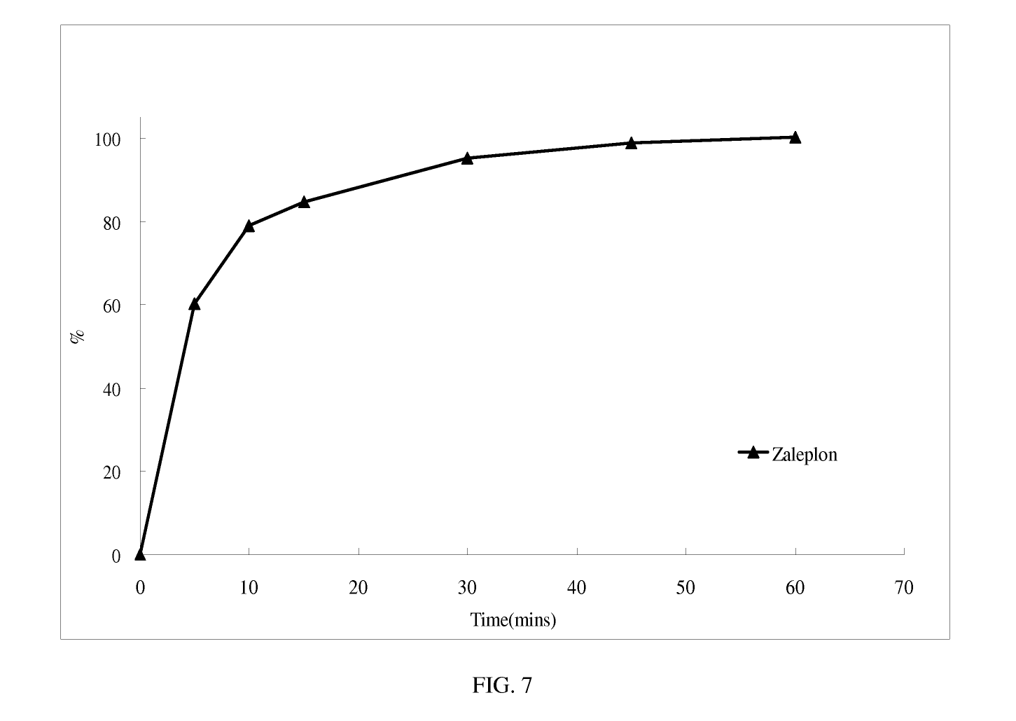

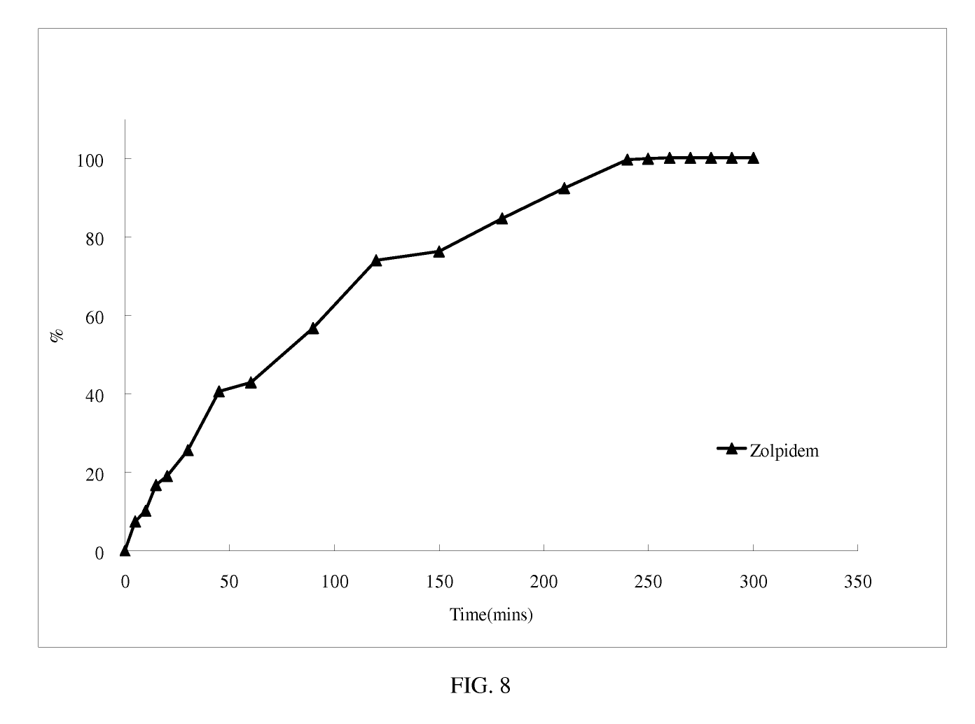

The dissolution results are also shown in FIG. 7. and FIG. 8. FIG. 7 shows the in vitro dissolution profile of the immediate release phase of Zaleplon, where over 80% Zaleplon dissolved in 15 minutes and the total amount of Zaleplon dissolved in 1 hour. FIG. 8 shows the in vitro dissolution profile of the sustained release phase of Zolpidem, where the total amount of Zolpidem dissolved in 5 hours.

What is claimed is:

1. A controlled-release formulation comprising Zaleplon or a pharmaceutically acceptable salt thereof in immediate release form and Zolpidem or a pharmaceutically acceptable salt thereof in sustained release form.

2. The controlled-release formulation of claim 1, wherein Zaleplon or a pharmaceutically acceptable salt thereof and Zolpidem or a pharmaceutically acceptable salt thereof are released in two phases, wherein according to a biphasic in vitro profile of dissolution when measured in a dissolution apparatus in about 0.1N hydrochloric acid buffer at about 37° C., the first phase is a immediate release phase of Zaleplon or a pharmaceutically acceptable salt thereof that is released more than about 70% within about 60 minutes, and the second phase is a sustained release phase of Zolpidem or a pharmaceutically acceptable salt thereof that is completely released between about 2 and about 6 hours.

3. The controlled-release formulation of claim 1, wherein Zaleplon is in free base form and Zolpidem is Zolpidem tartrate.

4. The controlled-release formulation of claim 1, wherein the amount of Zaleplon ranges from about 2 to about 10 mg.

5. The controlled-release formulation of claim 1, wherein the amount of Zaleplon ranges from about 2 to about 8 mg, about 2 to about 6 mg or about 3 to about 8 mg.

6. The controlled-release formulation of claim 1, wherein the amount of Zaleplon is about 5 mg.

7. The controlled-release formulation of claim 1, wherein the amount of Zolpidem ranges from about 3 to about 15 mg.

8. The controlled-release formulation of claim 1, wherein the amount of Zolpidem ranges from about 3 to about 12 mg, about 3 to about 10 mg, about 3 to about 8 mg, about 4 to about 12 mg, about 4 to about 10 mg, about 5 to about 15 mg or about 5 to about 10 mg.

9. The controlled-release formulation of claim 1, wherein the amount of Zolpidem is about 6.25 mg.

10. The controlled-release formulation of claim 1, wherein the sustained release form of Zolpidem comprises higher than about 5% by weight of a copolymer member selected from hydrogels, gelatin, polyethylene oxides; hydroxyalkylcelluloses; hydroxyethylcellulose, hydroxypropylcellulose; hydroxyisopropylcelluose; hydroxybutylcellulose; hydroxyphenylcellulose; hydroxyalkyl alkylcelluloses; hydroxypropyl methylcellulose; hydroxypropyl cellulose; hydroxypropylmethyl cellulose; polyethylene oxid poly(hydroxy alkyl methacrylate); poly(vinyl)alcohol; a mixture of methyl cellulose, cross-linked agar and carboxymethyl cellulose; a hydrogel; Carbopol™ acidic carboxy polymers; Cyanamer™ polyacrylamides; cross-linked water swellable indenemaleic anhydride polymers; Goodrite™ polyacrylic acid; starch graft copolymers; Aqua-Keeps™ acrylate polymer polysaccharides; and mixtures thereof

11. The controlled-release formulation of claim 10, wherein the sustained release form of Zolpidem comprises about 5% (w/w) to about 30% (w/w), about 5% (w/w) to about 25% (w/w), about 5% (w/w) to about 20% (w/w), about 5% (w/w) to about 15% (w/w), about 10% (w/w) to about 30% (w/w), about 10% (w/w) to about 25% (w/w), about 10% (w/w) to about 20% (w/w), about 10% (w/w) to about 15% (w/w), about 12% (w/w) to about 20% (w/w), about 12% (w/w) to about 17% (w/w) or about 12% (w/w) to about 15% (w/w) of a copolymer.

12. The controlled-release formulation of claim 1, wherein the sustained release Zolpidem is released between about 4 and about 6 hours.

13. The controlled-release formulation of claim 1, which is a tablet comprising a sustained release Zolpidem as a core coated with an immediate release layer of Zaleplon.

14. The controlled-release formulation of claim 13, wherein the sustained release Zolpidem is further coated with at least one release-slowing intermediate layer of polymethacrylate acrylic polymer.

15. The controlled-release formulation of claim 1, which is a double layer tablet comprising a layer of sustained release Zolpidem and a layer of immediate release Zaleplon.

16. The controlled-release formulation of claim 15, wherein the sustained release Zolpidem is further coated with at least one release-slowing intermediate layer of polymethacrylate acrylic polymer.

17. The controlled-release formulation of claim 1, which is a tablet with more than two layers comprising (i) one or two more layers of sustained release Zolpidem and (ii) one or two more layers of immediate release Zaleplon.

18. The controlled-release formulation of claim 17, wherein the sustained release Zolpidem is further coated with at least one release-slowing intermediate layer of polymethacrylate acrylic polymer.

19. The controlled-release formulation of claim 1, which is a capsule comprising a core pellet of sustained release Zolpidem coated with immediate release Zaleplon.

20. The controlled-release formulation of claim 19, wherein the sustained release Zolpidem is further coated with at least one water slightly soluble, release-slowing intermediate layer of high molecular polymer layer.

21. The controlled-release formulation of claim 1, which is a capsule comprising a pellet of sustained release entity of Zolpidem and a pellet of immediate release entity of Zaleplon.

22. The controlled-release formulation of claim 21, wherein the sustained release Zolpidem is further coated with at least one water slightly soluble, release-slowing intermediate layer of high molecular polymer layer.

23. The controlled-release formulation of claim 1, which is a capsule comprising a number of beads; each bead comprises a sustained release Zolpidem as a core coated with a immediate release layer of Zaleplon

24. The controlled-release formulation of claim 23, wherein the sustained release Zolpidem is further coated with at least water slightly soluble, release-slowing intermediate layer of high molecular polymer layer.

25. The controlled-release formulation of claim 1, which is used for preventing or treating sleep disorders selected from disorders associated with difficulties in staying asleep and/or falling asleep such as insomnia (e.g., transient, short-term, and chronic), delayed sleep phase syndrome, hypnotic-dependent sleep disorder, and stimulant-dependent sleep disorder; disorders associated with difficulties in staying awake such as sleep apnea, narcolepsy, restless leg syndrome, obstructive sleep apnea, central sleep apnea, idiopathic hypersomnia, respiratory muscle weakness-associated sleep disorder; disorders associated with difficulties in adhering to a regular sleep schedule such as sleep state misperception, shift work sleep disorder, chronic time zone change syndrome, and irregular sleep-wake syndrome; disorders associated with abnormal behaviors such as sleep tenor disorder (i.e., parasomnia) and sleepwalking (i.e., somnambulism); and other disorders such as sleep bruxism, fibromyalgia, and nightmares.
